(19) United States
(12) Patent Application Publication
(10) Pub. No.: US 2008/0045919 A1
(43) Pub. Date: Feb. 21, 2008

(54) Liquid Transfer Device for Medical Dispensing Containers

(75) Inventors: Laurent Jakob, Bernex-Suisse (CH); Frederic Rivas, Bonneville (FR)

Correspondence Address: BRACCO RESEARCH USA INC., 305- COLLEGE ROAD EAST, PRINCETON, NJ, 08540, US

(73) Assignee: Bracco Research S.A., Plan-les-Ouates (CH)

(21) Appl. No.: 11/794,024

(22) PCT Filed: Dec. 20, 2005
(86) PCT No.: PCT/EP05/56975

§ 371 Date: Jun. 21, 2007

(30) Foreign Application Priority Data
Dec. 23, 2004 (EP) 04106959.2

(51) Int. Cl. A61B 19/00

(52) U.S. Cl. 604/406; 604/416

(57) ABSTRACT

Liquid transfer device to be used to transfer a liquid into or from a dispensing container, particularly a medical dispensing container like a vial for diagnostic agents and kit comprising a vial, a syringe and a device of the aforementioned type. The transfer device comprises a valve which substantially limits the gaseous exchange between the inside of a vial and the external ambient, when the device is connected to the vial in steady state conditions.

### TECHNICAL FIELD

The invention relates to a liquid transfer device to be used to transfer a liquid into or from a medical dispensing container, such as a vial for diagnostic agents, and particularly to a pharmaceutical kit comprising said device and said container.

### BACKGROUND OF THE INVENTION

Medical dispensing containers made of glass or polymeric materials, the walls of which are non-collapsible, typically require an air inlet when a medical fluid is withdrawn therefrom, to prevent the formation of vacuum therein. Typically, vials containing a medical fluid are closed by rubber stoppers which are pierced by a spike of a transfer device having a duct for the passage of the medical fluid and a ventilation duct. Examples of devices comprising a liquid fluid duct and a ventilation duct are disclosed, for instance, in U.S. Pat. No. 3,797,521, U.S. Pat. No. 4,262,671, U.S. Pat. No. 4,623,343, U.S. Pat. No. 4,857,068, U.S. Pat. No. 5,041,106, and U.S. Pat. No. 6,139,534.

The present invention is particularly concerned with the liquid transfer into a container containing a medicament reconstitutable upon addition of said liquid, and the subsequent removal of the reconstituted medicament from said container. More particularly, the device of the invention is suitable for the preparation and dispensing of some diagnostic or therapeutic agents, such as those comprising a gaseous component including, for instance, gas-filled microvesicles for ultrasound diagnostic and/or therapeutic use.

Gas-filled microvesicles for ultrasound diagnostic and/or therapeutic use include suspensions of gas bubbles having a diameter of a few microns dispersed in an aqueous medium. Of particular interest are gas bubbles which are stabilized by means of suitable additives such as, for example emulsifiers, oils, thickeners or sugars, or by entrapping or encapsulating the gas or a precursor thereof in a variety of systems. These agents are designed to be used primarily as intravenous or intra-arterial injectables in conjunction with the use of medical echographic equipment which employs for example, B-mode image formation (based on the spatial distribution of backscatter tissue properties) or Doppler signal processing (based on Continuous Wave or pulsed Doppler processing of ultrasonic echoes to determine blood or liquid flow parameters).

A first category of stabilized bubbles or microvesicles is generally referred to in the art as “microbubbles” and includes aqueous suspensions in which the bubbles of gas are bounded at the gas/liquid interface by a very thin envelope (film) involving a stabilizing amphiphilic material disposed at the gas to liquid interface. Microbubble suspensions are typically prepared by contacting powdered amphiphilic materials, e.g. freeze-dried preformed liposomes or freeze-dried or spray-dried phospholipid solutions, with air or other gas and then with an aqueous carrier, while agitating to generate a microbubble suspension which can then be administered.

Examples of aqueous suspension of gas microbubbles and preparation thereof are disclosed, for instance, in U.S. Pat. No. 5,271,928, U.S. Pat. No. 5,445,813, U.S. Pat. No. 5,413,774, U.S. Pat. Nos. 5,556,610, 5,597,549, U.S. Pat. No. 5,827,504, WO 97/29783 and WO 04/069284.

Commercially available ultrasound contrast agents of this type include for instance SonoVue® (Bracco International BV).

A second category of microvesicles is generally referred to in the art as “microballoons” or “microcapsules” and includes suspensions in which the bubbles of gas are surrounded by a solid material envelope of a lipid or of natural or synthetic polymers. Examples of microballoons and of the preparation thereof are disclosed, for instance, in U.S. Pat. No. 5,711,933 and U.S. Pat. No. 6,333,021.

Whilst the above formulations are administered as suspensions of gas-filled microvesicles in a suitable physiologically acceptable liquid, for storage purposes it is in general preferred to use precursors of said microvesicles in dry (e.g. lyophilized) form, as disclosed in the above mentioned patents and patent applications. The microvesicles suspension is then obtained by adding to said dry precursors, in the presence of a suitable gas (e.g a fluorinated gas), a physiologically acceptable liquid carrier, preferably under agitation. The dry precursor can for instance be stored in a vial (e.g. of glass) in the presence of a desired gas, said vial being sealed with a suitable stopper (e.g. of rubber), through which the liquid carrier can be injected. The contrast agent formulation can thus be supplied in a kit comprising a vial (containing the dry precursor and the gas) and a pre-filled syringe (containing the physiologically acceptable liquid carrier). The syringe can be associated with a suitable liquid transfer device which typically comprises means for piercing the stopper, for injecting the liquid carrier into the vial and withdrawing the formed microbubbles suspension from it, and for allowing a gas/air flow from and into the container during the respective liquid injection and withdrawal phases. This transfer device is preferably comprised in the kit as a separate device connectable (e.g. through a luer lock) to the syringe. Examples of such devices are disclosed, for instance, in U.S. Pat. No. 6,743,214.

When the suspension of gas-filled microbubbles has been reconstituted with the addition of the liquid, it may however be desirable to keep said reconstituted suspension in the vial for a relatively long time (e.g. one for few hours). As observed by the Applicant, such a relatively long storage time of the reconstituted suspension may however pose some problems, particularly in connection with a possible exchange between the gas contained inside the container and the outer atmosphere air. This may be for instance the case when a liquid transfer device (such as the one disclosed in U.S. Pat. No. 6,743,214) is employed, where a direct fluid-gas passage is present between the inside of the container and the outer ambient, with consequent possible air inlet inside the container. While it has been demonstrated that a fluorinated gases employed for filling the microvesicles can be admixed with relatively high amounts of air (e.g. up to 70-80% by volume of air) without substantially modifying the properties and stability of the gas-filled microvesicles (see e.g. EP patent no. 682 530), an excessive amount of air may nevertheless negatively affect said properties and stability. In addition, when the gas filled microvesicles already contain a mixture of fluorinated gas and air (as in the above mentioned EP 682 530), the negative effects deriving by said air inlet may be more evident.

Furthermore, the above undesirable gas/air exchange may similarly take place also when the transfer device is connected to the vial and left in place for a certain time, without connecting a syringe thereto and/or injecting a liquid into the vial.

Having perceived the above problem, the Applicant has thus devised a liquid transferring device provided with a suitable valve capable of substantially limit the gaseous exchange between said container and the ambient atmosphere. Furthermore, as observed by the Applicant, due to the relative sensitivity of gas-filled microbubbles to excessively high or negative pressures, the pressure inside the container shall preferably be controlled during injection or withdrawal phases of a liquid from the container. For this reason, the valve of the liquid transfer device of the invention is preferably adapted to control the internal pressure of the container during the liquid injection or withdrawal phases.

Liquid transfer devices provided with valves are already known in the art, such as those described in U.S. Pat. No. 3,797,521, U.S. Pat. No. 4,857,068 and U.S. Pat. No. 5,041,106. Said check valves are however essentially one-way valves, for allowing an inlet flow of air when a liquid is withdrawn from the container. No means are foreseen in said prior art valves for allowing/controlling overpressure gas discharge upon liquid injection. FR 2580931 discloses means for protecting the filter and regulating the pressure in a liquid transfer device. Said means allow an air inlet into a container connected to the transfer device (upon liquid removal from said container) at a pressure of about 10 mbar and an air outlet (upon liquid injection into the container) at pressures higher than 300 mbar.

### SUMMARY OF THE INVENTION

A first aspect of the present invention relates to pharmaceutical kit for the preparation of a medicament comprising:

a) a medical dispensing container defining an inner space and containing therein a diagnostically and/or therapeutically effective agent in the form of a physiologically acceptable gas;

b) a liquid transfer device, which cooperates with said container for transferring a liquid into or from said container, said device comprising a first and a second conduit, said first and said second conduit allowing a fluid contact between the inner space and an external ambient when said device cooperates with said container, said second conduit comprising a valve, wherein:

when said device cooperates with the container during a liquid flow into or from said dispensing container, said liquid flow is effected through said first conduit while said valve allows a gaseous flow between said container and an external ambient through second conduit; and

when said device cooperates with the container in steady state conditions, said valve substantially prevents a gaseous exchange between said container and the external ambient.

Said valve is preferably adapted to permit a gaseous flow from the container towards the external ambient when the value of pressure within the container reaches a predetermined maximum value of overpressure or a predetermined minimum value of negative pressure. Said values of overpressure and/or of negative pressure (jointly referred to as “differential” pressure) are preferably selected such that said respective values do not negatively affect the stability of a medicament contained in the container. Preferably said values of differential pressures are lower than 300 mbar, more preferably lower than 150 mbar and even more preferably lower than 100 mbar.

Advantageously the transfer device comprises a filter associated to the second conduit, for protecting the content of the vial against microbial contamination during liquid withdrawal. Preferably, said filter also prevents the fluid to flow out from the container into the external ambient. Said filter is preferably a liquid impermeable/gas permeable filter. More preferably it is a hydrophobic filter.

According to a preferred embodiment said transfer device comprises a connector for connecting a fluid injector, such as a syringe, thereto. Preferably, said connector is a luer connector.

Preferably the transfer device comprises a valve associated to first conduit for closing the conduit when the fluid injector is not connected thereto. The medical container is preferably a vial, typically having a substantially a cylindrical body, a flat bottom portion and a top portion defining an open area closed by a stopper hermetically sealing the content of the vial. The vial's content can be, for instance, a suspension of gas-filled microvesicles for diagnostic and/or therapeutic use or a precursor thereof, e.g. in the form of a dry lipid deposit, in contact with a physiologically acceptable gas.

A further aspect of the invention relates to an assembly comprising a liquid transfer device and a medical container as above defined.

A further aspect of the invention relates to a pharmaceutical kit comprising a liquid transfer device as above defined, a vial containing a pharmaceutically active formulation (e.g. a dry lipid deposit and a physiologically acceptable gas) and a syringe prefilled with a physiologically acceptable (e.g. saline) solution. The liquid transfer device is used for injecting the solution into the vial and withdrawing the reconstituted medicament.

The characteristics of the invention and the advantages derived therefrom will appear more clearly from the following description of non limiting embodiments, illustrated in the annexed drawings.

### BRIEF DESCRIPTION OF THE DRAWINGS

FIG. 1 shows a cross sectional view of a first embodiment of a liquid transfer device according to the invention;

FIG. 2a shows a cross sectional view of a second embodiment of a liquid transfer device according to the invention;

FIG. 2b shows a simplified cross sectional view of a valve useful for a liquid transfer device according to the invention;

FIG. 3a-3c schematically illustrates the phases involved in the preparation of a diagnostic agent with a liquid transfer device according to the invention;

FIG. 4 shows the corresponding pressure diagram of the preparation steps illustrated in FIGS. 3a-3c;

FIG. 5 shows the diagram of air diffusion as a function of time, measured for different liquid transfer devices.

FIGS. 6a and 6b show the pressure diagram associated with a liquid injection and withdrawal by using transfer device of the invention.

### DETAILED DESCRIPTION OF THE INVENTION

The liquid transfer device associated to a medical container in a kit according to the invention comprises a valve which allows a gaseous flow between a container and an external ambient (when a liquid is introduced or withdrawn from said container), while substantially preventing any gaseous flow into said container from said external ambient in steady state conditions. In particular, the valve allows a first gaseous flow (from the container towards the external ambient) when the pressure inside the contained exceeds a certain value, and a second gaseous flow (from the external ambient towards the container) when the pressure inside the container falls below a certain value of negative pressure. In particular, according to the present invention, the substantial avoidance of gas exchange can be defined as a gaseous exchange which is preferably of less than about 25% (v/v) of the total volume of the container during a period of time of 6 hours, more preferably of less than about 20% and even more preferably of less than 15%.

The term valve as used herein includes either a single valve which allows a gaseous flow in both directions also known as “bi-directional valves”, or a combination of two or more valves, typically mono-directional; in this latter case, a typical configuration is a combination of a first mono-directional valve, which allows a flow of gas only in a first direction (e.g. from the container to the external ambient) and a second mono-directional valve, which allows a flow of gas only in the reverse direction.

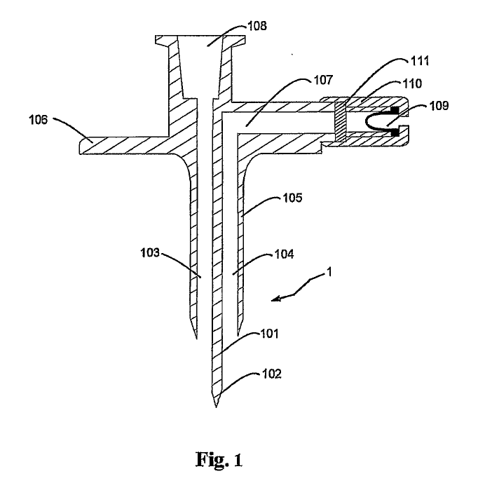

With reference to FIG. 1, a first embodiment of the invention is shown. The liquid transfer device 1 comprises a spike 101 having a sharp end 102 for piercing the closure of a dispensing container, a liquid fluid passage 103 and a gas passage 104 opening at a tip. The spike 101, formed as an integral member with the fluid 103 and gas passages 104, carries a body member 105 that includes a handle 106 and a chamber 107 extending radially outwardly from the spike 101. The spike 101 has a tubular extension rearward of the body member 105. The fluid passage 103 extends substantially parallel to the longitudinal axis of the spike 101 throughout its length and ends with a luer connector 108, while the gas passage 104 extends parallel to the fluid passage 103 through the spike 101 and the body member 105, ending into a final portion 107 in the form of a chamber. A bi-directional valve 109 is housed in the final portion 107 of the gas passage 104 in a holder 110 that extends radially outwardly the body member 105 and is in communication with the external ambient. The valve 109 is advantageously a check valve such as the one described in U.S. Pat. No. 4,434,810, herein incorporated by reference. An example of a suitable commercial is 4VA5033 by Vernay Laboratories, Inc. A hydrophobic filter 111 is preferably provided before the valve, for protecting the content of the vial against possible microbial contamination (e.g. with the air-inlet during liquid withdrawal). The filter is also preferably adapted to avoid undesirable liquid leakage outside the vial, e.g. when the device-vial assembly is turned upside down for liquid removal. The hydrophobic filter has typical pore sizes of from about 0.20 μm to about 0.50 μm.

Any kind of bi-directional valve can be used as well as any combination of two or more mono-directional valves that, when the transfer device is connected to a container, permits a gaseous flow between the container and the external ambient when a liquid is introduced or withdrawn from the container, substantially preventing any gaseous flow into the container from the external ambient in steady state conditions.

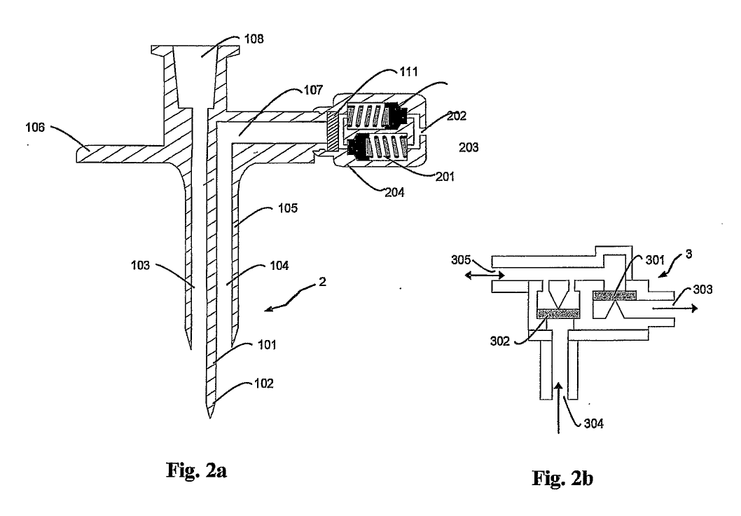

With reference to FIG. 2a, a second embodiment of the invention is shown. Here the check valve 109 is replaced by two one-way (or mono-directional) spring pressure valves 201, 202 in anti-parallel assembly. The two spring valves 201, 202 of FIG. 2a are housed in a holder 204 at the end of the final portion 107 of the gas passage 104 through the hydrophobic filter 111 and are in communication with the external ambient via an opening 203 in the holder 204. The two valves 201 and 202 have mutually opposite orientations for allowing an inlet and outlet flow. Examples of suitable spring valves are disclosed for instance in U.S. Pat. No. 5,349,984, here incorporated by reference. An example of a suitable commercial valve is check valve ref. 23602001 (Halkey-Roberts). Alternatively, a combined membrane valve 3 as schematically depicted in FIG. 2b can be used in place of the two spring valves. Said valve comprises two mono-directional membrane valves 301 and 302, having mutually opposite orientation, associated with respective separate conduits for inlet 304 and outlet 303 gas-flow. Said conduits are placed between a common inlet/outlet conduit 305 and the respective outlet 303 and inlet conduit 304. The common conduit 305 can be connected at the end of the final portion 107 of the gas passage 104 of the spike 2 through the hydrophobic filter ill while the conduits 303 and 304 can be placed in communication with the external ambient either directly or through a common connection (not shown). For instance, commercial valve #798269 by Sidam can advantageously be used.

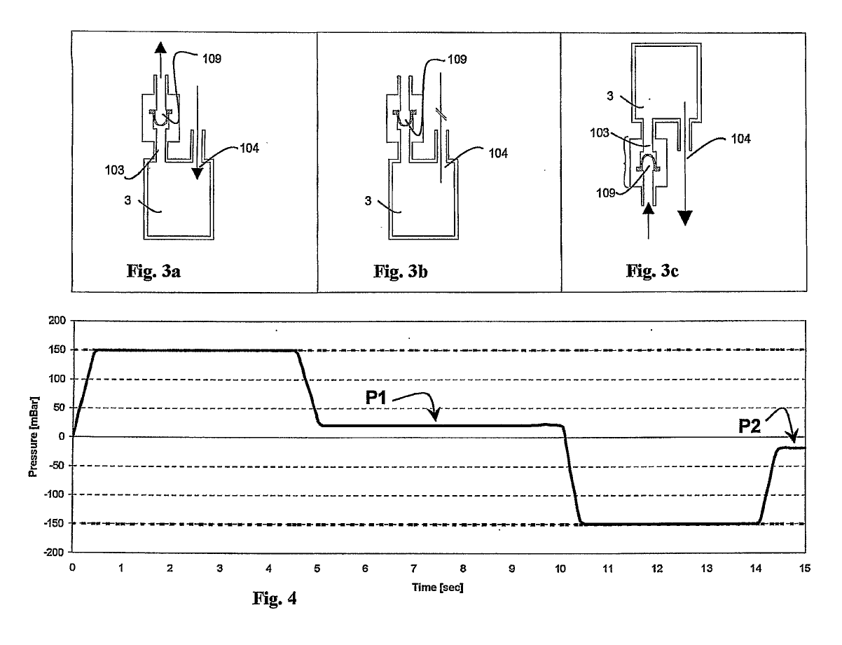

The upper part of FIG. 3 (FIGS. 3a-3c) schematically illustrates the steps involved in the preparation of a pharmaceutically active formulation by using a transfer device according to the invention. The transfer device is used for injecting a physiologically acceptable liquid carrier in a vial containing a precursor of a pharmaceutically active formulation for reconstitution thereof. The pharmaceutically active formulation can be for instance a suspension of gas-filled microvesicles which is reconstituted from a dry powder (e.g. comprising a phospholipid) deposited on the bottom of the vial, in contact with a physiologically acceptable gas (e.g. a perfluorinated gas, such as sulfur hexafluoride or perfluorobutane). The vial and the two conduits of the transfer device are schematically depicted as a box 3 with two channels, one for the injection/removal of the fluid 104 and one for allowing the gaseous flow 103 between the vial 3 and the external ambient through a bi-directional valve 109 of the check type. FIG. 4 shows a schematic pressure diagram illustrating the variation of pressure occurring during the three steps of FIGS. 3a-3c.

In particular, when a forward flow is caused by the injection of a liquid carrier into the vial (step 1, FIG. 3a), the pressure inside the container suddenly increases up to a certain value (e.g. about 150 mbar in FIG. 4), which depends typically from the injection speed and from the dimensions of the ventilation duct, and to a lesser extent from the form and dimension of the container and from the predetermined value of pressure at which the valve is set to open. As regards to the opening pressure of the valve, it should be noted that, while the valve opens when the overpressure inside the container reaches a predetermined value to allow the gas under pressure to be expelled from the container through the ventilation duct (“opening overpressure”, about 20 mbar in the schematic example of FIG. 4), the overpressure inside the container may nevertheless reach relatively higher values, which are mainly determined by a reduced dimension of the ventilation duct and by a relatively high injection speed. When the injection is effected at a substantially constant (high) rate, the overpressure inside the container typically remains relatively constant until substantially the whole injection phase is completed. When all the liquid carrier has been injected into the vial and the first conduit has been sealed, the overpressure in the container gradually reduces at a value substantially corresponding to the predetermined value of opening (or closing) pressure of the valve. At this point, when a steady-state condition is reached, the valve closes. One end of the valve is thus exposed at the internal pressure of the vial while the other end is exposed at the external ambient pressure, the differential pressure on the valve being P1. In the simplified example of the figure P1 is 20 mBar, i.e. the valve is supposed to allow a forward flow of gas from the container to the external ambient for differential pressures equal or greater than 20 mBar. If the content is not removed from the vial and the entrance of the liquid channel is closed (step 2), substantially no air flows inside the vial. In general, the higher the value of P1, the lower the air leakage into the container. However, too high values of P1 should in general be avoided. A first reason is that, if P1 is excessively high (i.e. if the valve opens at high values of pressure), this may determine excessively high temporary overpressure values during the injection phase of the liquid carrier, which may negatively affect the stability of the vial's content, in particular when the vial contains a suspension of gas-filled microvesicles. Another reason is to avoid keeping the content of the vial under relatively elevated overpressure values during the steady-state phase (which may last for few hours); as a matter of fact, even relatively low overpressures, which can in general be tolerated by a substance for short periods of times, may negatively affect the stability of said substance when applied for long periods of time. Accordingly, the valve contained in the transfer device is preferably selected to open at a value of overpressure in the container (defined as differential pressure between the inside of the container and the outer ambient pressure) of less than 300 mbar, preferably of less than 150 and much more preferably at a value of less than 100 mbar. To keep a certain overpressure inside the container under steady state conditions, said opening pressure shall however preferably be of at least 10 mbar or higher, more preferably of at least 20 mbar. A value of about 50 mbar is particularly preferred.

During step 3 (FIG. 3c) the vial is turned upside-down and the reconstituted agent is withdrawn from the vial. When a reverse flow is caused by the aspiration through the first conduit, the pressure inside the container decreases down to a certain negative value (about −150 mbar in FIG. 4), which again typically depends mainly from the withdrawal speed and the dimensions of the ventilation duct; during the liquid withdrawal phase, the valve will open when a predetermined negative pressure is reached inside the container, in order to allow air to enter through the ventilation duct and compensate the negative pressure. Similarly to the injection phase, when the liquid is withdrawn form the container at a substantially constant rate, the negative pressure inside the container typically remains substantially at said value during the withdrawal of the liquid. When all the content has been withdrawn from the vial, the negative pressure inside the vial is gradually reduced (in absolute value), to reach a final value corresponding substantially to the value of opening of the valve (“opening negative pressure”, about −20 mbar in FIG. 4), for allowing a reverse gaseous flow from the external ambient into the vial. At this point the valve closes again. The valve is thus submitted to a differential pressure P2, defined by the difference between the external ambient pressure and the pressure inside the vial. In this simplified example P2 is 20 mBar, i.e. the valve is supposed to allow a reverse flow of air from the external ambient to the container for differential pressures higher than 20 mBar. Similarly, the valve is preferably selected to open at a value of negative pressure inside the container (defined as the differential pressure between the outer ambient pressure and the pressure inside of the container) of about 300 mbar or lower, preferably of about 150 or lower and much more preferably at a value of 100 or lower. As above, said opening pressure is preferably be of at least 10 mbar or higher, more preferably of at least 20 mbar. The transfer device of the invention can advantageously be used for injecting and/or withdrawing a liquid into and/or from a container which contains a pharmaceutically active formulation comprising a gaseous material. The use of the transfer device is particularly advantageous when the gaseous material is a diagnostically and/or therapeutically effective agent, such as gaseous materials employed for the preparation of gas-filled microvesicles for use in ultrasound diagnostic and/or therapeutic methods.

The term “pharmaceutically active formulation” includes within its meaning any formulation, or precursor thereof, capable of exerting a pharmaceutical effect when administered in an effective amount, including diagnostic and/or therapeutic effects. Examples of pharmaceutically active formulations are those formulations which comprise, for instance, a diagnostic agent and/or a bioactive agent.

The term “diagnostic agent” includes within its meaning any compound, composition or particle which may be used in connection with methods for imaging an internal region of a patient and/or diagnosing the presence or absence of a disease in a patient. Exemplary diagnostic agents include, for example, contrast agents for use in connection with magnetic resonance imaging, X-ray imaging, in particular computed tomography, optical imaging, nuclear imaging or molecular imaging of a patient including, for example, magnetite nanoparticles.

The term “bioactive agent” includes within its meaning any substance, composition or particle which may be used in any therapeutic application, such as in methods for the treatment of a disease in a patient, as well as any substance which is capable of exerting or responsible to exert a biological effect in vitro and/or in vivo. Examples of bioactive agents are drugs, medicaments, pharmaceuticals, proteins, natural or synthetic peptides, including oligopeptides and polypeptides, vitamins, steroids and genetic material, including nucleosides, nucleotides and polynucleotides. In particular, said container can be a vial (e.g. of glass) containing a suspension of gas-filled microvesicles, such as those disclosed in the above mentioned documents, U.S. Pat. No. 5,271,928, U.S. Pat. No. 5,445,813, U.S. Pat. No. 5,413,774, U.S. Pat. Nos. 5,556,610, 5,597,549, U.S. Pat. No. 5,827,504, WO 97/29783, WO 04/069284, U.S. Pat. No. 5,711,933 and U.S. Pat. No. 6,333,021, all herein incorporated by reference. According to a preferred embodiment, said vial comprises a precursor of said microvesicles in the form of a dry powdered deposit in contact with a physiologically acceptable gas. Preferably the microvesicles, or their precursor, are gas-filled microbubbles stabilized by a layer of amphiphilic material. Preferably said amphiphilic material comprises a phospholipid, such as fatty acids di-esters of phosphatidylcholine, ethylphosphatidylcholine, phosphatidylglycerol, phosphatidic acid, phosphatidyl-ethanolamine, phosphatidylserine, sphingomyelin or mixtures thereof. Examples of preferred phospholipids are, for instance, dilauroyl-phosphatidylcholine (DLPC), dimyristoyl-phosphatidylcholine (DMPC), dipalmitoyl-phosphatidylcholine (DPPC), diarachidoyl-phosphatidylcholine (DAPC), distearoyl-phosphatidylcholine (DSPC), dioleoyl-phosphatidylcholine (DOPC), 1,2 Distearoyl-sn-glycero-3-Ethylphosphocholine (Ethyl-DSPC), dipentadecanoyl-phosphatidylcholine (DPDPC), 1-myristoyl-2-palmitoyl-phosphatidylcholine (MPPC), 1-palmitoyl-2-myristoyl-phosphatidylcholine (PMPC), 1-palmitoyl-2-stearoyl-phosphatidylcholine (PSPC), 1-stearoyl-2-palmitoyl-phosphatidylcholine (SPPC), 1-palmitoyl-2-oleylphosphatidylcholine (POPC), 1-oleyl-2-palmitoyl-phosphatidylcholine (OPPC), dilauroyl-phosphatidylglycerol (DLPG) and its alkali metal salts, diarachidoylphosphatidyl-glycerol (DAPG) and its alkali metal salts, dimyristoylphosphatidylglycerol (DMPG) and its alkali metal salts, dipalmitoylphosphatidylglycerol (DPPG) and its alkali metal salts, distearoylphosphatidylglycerol (DSPG) and its alkali metal salts, dioleoyl-phosphatidylglycerol (DOPG) and its alkali metal salts, dimyristoyl phosphatidic acid (DMPA) and its alkali metal salts, dipalmitoyl phosphatidic acid (DPPA) and its alkali metal salts, distearoyl phosphatidic acid (DSPA), diarachidoylphosphatidic acid (DAPA) and its alkali metal salts, dimyristoyl-phosphatidylethanolamine (DMPE), dipalmitoylphosphatidylethanolamine (DPPE), distearoyl phosphatidyl-ethanolamine (DSPE), dioleylphosphatidyl-ethanolamine (DOPE), diarachidoylphosphatidyl-ethanolamine (DAPE), dilinoleylphosphatidylethanolamine (DLPE), dimyristoyl phosphatidylserine (DMPS), diarachidoyl phosphatidylserine (DAPS), dipalmitoyl phosphatidylserine (DPPS), distearoylphosphatidylserine (DSPS), dioleoylphosphatidylserine (DOPS), dipalmitoyl sphingomyelin (DPSP), and distearoylsphingomyelin (DSSP).

The term phospholipid further includes modified phospholipid, e.g. phospholipids where the hydrophilic group is in turn bound to another hydrophilic group. Examples of modified phospholipids are phosphatidylethanolamines modified with polyethylenglycol (PEG), i.e. phosphatidylethanolamines where the hydrophilic ethanolamine moiety is linked to a PEG molecule of variable molecular weight e.g. from 300 to 5000 daltons), such as DPPE-PEG or DSPE-PEG, i.e. DPPE (or DSPE) having a PEG polymer attached thereto. For example, DPPE-PEG2000 refers to DPPE having attached thereto a PEG polymer having a mean average molecular weight of about 2000.

The phospholipids can optionally be admixed with other lipids, such as cholesterol, ergosterol, phytosterol, sitosterol, lanosterol, tocopherol, propyl gallate or ascorbyl palmitate, fatty acids such as myristic acid, palmitic acid, stearic acid, arachidic acid and derivatives thereof.

Bulking agents, having cryoprotective and/or lyoprotective effects, can also be added to the composition, such as, for instance, an amino-acid such as glycine; a carbohydrate, e.g. a sugar such as sucrose, mannitol, maltose, trehalose, glucose, lactose or a cyclodextrin, or a polysaccharide such as dextran; or a polyglycol such as polyethylene glycol.

Any biocompatible gas, gas precursor or mixture thereof may be employed to fill the above microvesicles.

In the present description and claims, the term “biocompatible” or “physiologically acceptable” refers to any compound, material or formulation (in solid, liquid or gaseous form) which can be administered, in a selected amount, to a patient without negatively affecting or substantially modifying its organism's healthy or normal functioning (e.g. without determining any status of unacceptable toxicity, causing any extreme or uncontrollable allergenic response or determining any abnormal pathological condition or disease status).

Suitable gases may comprise, for example nitrogen; oxygen; carbon dioxide; hydrogen; nitrous oxide; a noble or inert gas such as helium, argon, xenon or krypton; a radioactive gas such as Xe¹³³ or Kr⁸¹; a hyperpolarized noble gas such as hyperpolarized helium, hyperpolarized xenon or hyperpolarized neon; a low molecular weight hydrocarbon (e.g. containing up to 7 carbon atoms), for example an alkane such as methane, ethane, propane, butane, isobutane, pentane or isopentane, a cycloalkane such as cyclobutane or cyclopentane, an alkene such as propene, butene or isobutene, or an alkyne such as acetylene; an ether; a ketone; an ester; halogenated gases, preferably fluorinated gases, such as or halogenated, fluorinated or perfluorinated low molecular weight hydrocarbons (e.g. containing up to 7 carbon atoms); or a mixture of any of the foregoing. Where a halogenated hydrocarbon is used, preferably at least some, more preferably all, of the halogen atoms in said compound are fluorine atoms.

Fluorinated gases are preferred, in particular perfluorinated gases, especially in the field of ultrasound imaging. Preferred compounds are perfluorinated gases, such as SF₆ or perfluorocarbons (perfluorinated hydrocarbons), i.e. hydrocarbons where all the hydrogen atoms are replaced by fluorine atoms. The term perfluorocarbon includes saturated, unsaturated, and cyclic perfluorocarbons. Suitable perfluorocarbons include, for example, CF₄, C₂F₆, C₃F₈, C₄F₈, C₄F₁₀, C₅F₁₂, C₆F₁₂, C₆F₁₄, C₇F₁₄, C₇F₁₆, C₈F₁₈, and C₉F₂₀; preferably C₃F₈, C₄F₁₀ or C₅F₁₂ are employed, optionally in admixture with air nitrogen.

The gas is typically introduced in the container containing the lyophilized precursor of microvesicles at about atmospheric pressure (i.e. about 1020 mbar ±5%) or at a pressure lower than the atmospheric one (e.g. 900 mbar or lower) as disclosed e.g. in European patent application EP 1228770. The container is then typically sealed by a gas-seal stopper, preferably made from an elastomeric compound or multicomponent formulation based on an elastomer, such as poly(isobutylene) or butyl rubber. Conveniently, a butyl rubber stopper from Daiko Seiko ltd. can be used.

Microvesicles suspensions are then formed by introduction of a suitable physiologically acceptable liquid carrier into the container followed by agitation of the mixture to reconstitute an injectable composition.

Suitable physiologically acceptable liquid carriers are sterile water, aqueous solutions such as saline (which may advantageously be balanced so that the final product for injection is not hypotonic), or solutions of one or more tonicity adjusting substances such as salts or sugars, sugar alcohols, glycols or other non-ionic polyol materials (e.g. glucose, sucrose, sorbitol, mannitol, glycerol, polyethylene glycols, propylene glycols and the like).

According to a preferred aspect of the invention, the kit further comprises a physiologically acceptable liquid carrier, for reconstituting the suspension of gas-filled microvesicles. The liquid carrier is preferably contained into a separate container (typically in the form of a syringe) which is used for injecting the liquid carrier into the container and for withdrawing the reconstituted suspension therefrom, through the liquid transfer device. Although in general hand shaking of the container provides the desired energy for reconstituting the suspension, means for directing or permitting application of sufficient energy towards the container can also be provided (e.g. a Vortex mixer), in order to assure suitable reconstitution of the suspension.

The liquid transfer device as above defined can thus be used for reconstituting a suspension of gas-filled microvesicles, by connecting a syringe to the liquid transfer device, introducing the physiologically acceptable liquid of the syringe into the vial containing the dry powdered precursor of said gas-filled microbubbles (in contact with the desired gas), agitating the content of the vial and withdrawing the obtained suspension.

Due to the improved characteristics of the liquid transfer device, the phase of removal of the suspension can be performed several hours or days after the reconstitution of the pharmaceutically active formulation, without substantially altering the content of gas/air of the reconstituted formulation.

### EXAMPLES

### Example 1

### Measure of Gas Exchange with Different Valves

18 vials (each having an internal volume of about 11 cm³) are filled with SF₆ gas at room temperature up to about atmospheric pressure and sealed with a rubber stopper.

The vials are divided in three groups of six vials each and the stopper of each vial is then pierced with a liquid transfer device as indicated hereinafter:

Device 1a (comparative): device of FIG. 1, with clogged liquid duct and without valve 109;

Device 1b: device of FIG. 1, with clogged liquid duct and valve Vernay 5033;

Device 1c: device of FIG. 2, with clogged liquid duct and two valves Halkey-Roberts 246302001.

For all the devices, the diameter of the ventilation duct is of about 1.1 mm.

Groups of six devices for each type are used for piercing respective groups of six vials. The six vials of each group are then used to determined the content of air penetrated therein as a function of time, by removing the device from the vials after 1, 2, 3, 4, 5 or 6 hours, respectively, sealing the vial and measuring the residual concentration of SF₆ by means of a gas-chromatograph Hewlett-Packard GC 6890 equipped with a Hewlett-Packard Headspace injector and TCD detector (capillary column: Chrompack plot Fused silica 25 m×0.32 mm coating Poraplot Q)

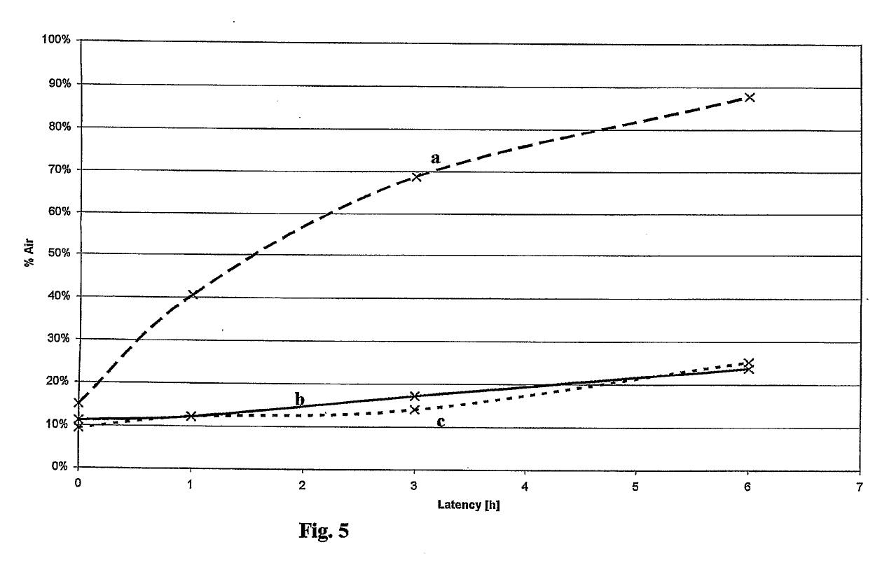

The difference to 100% gives the amount of air penetrated in the vial. FIG. 5 shows the results of the experiment, indicating that the presence of suitable valves in the transfer device (lines b and c) allow to substantially limit the penetration of air inside the vial with respect to a device without valve (line a).

### Example 2

### Measure of Pressure Variation During Liquid Injection

To measure the pressure variation inside a vial upon liquid injection and withdrawal by using a transfer device according to example 1b, the following set-up has been used.

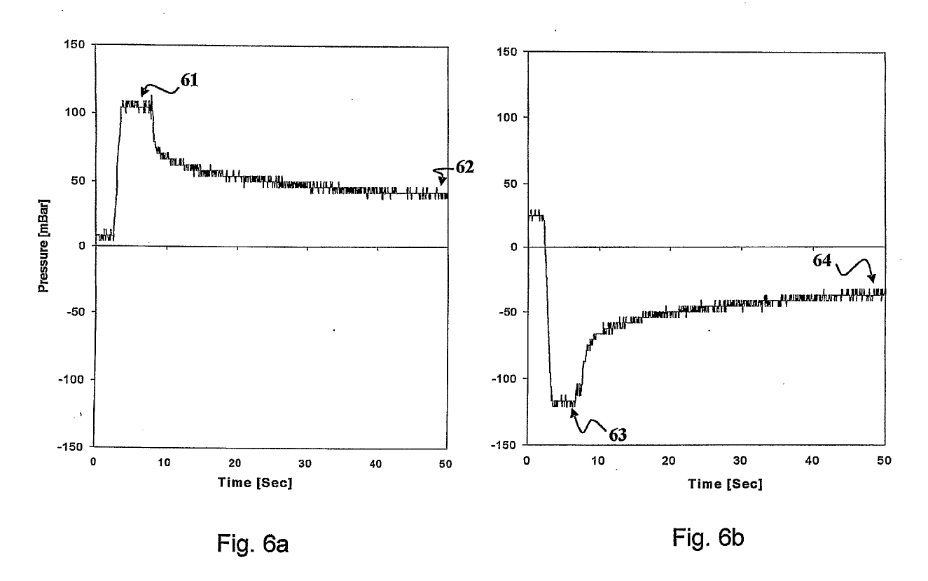

A syringe filled with saline solution is attached to a duct comprising a calibrated differential pressure transducer (COBE® pressure sensor Ref: #041-500-5003) and then attached to the liquid duct of the device according to example lb. The whole assembly is then inserted in a vial as described in example 1. The syringe plunger is moved at constant speed to allow a substantially constant injection rate (1 ml/sec) in the vial, for a total volume of 2 ml. FIG. 6a shows the variation of pressure inside the vial as a function of time: (61) correspond to the injection step and (62) to the intermediate steady state. The injected solution is left in the vial for about 50 seconds. Afterwards, the vial is inverted and the solution is withdrawn therefrom. FIG. 6b shows the variation of pressure inside the vial as a function of time: (63) correspond to the withdrawal step and (64) to the final steady state.

1. A pharmaceutical kit for the preparation of a medicament comprising:
a) a container defining an inner space and containing therein a diagnostically and/or therapeutically effective agent in the form of a physiologically acceptable gas; and
b) a liquid transfer device, which cooperates with said container for transferring a liquid into or from said container, said device comprising a first and a second conduit, said second conduit comprising a valve, wherein:
when said device cooperates with the container during liquid flow into or from said container, said liquid flow is effected through said first conduit while said valve allows a gaseous flow between said container and said external ambient through said second conduit; and
when said device cooperates with the container in steady state conditions, said valve substantially prevents a gaseous exchange between said container and the external ambient, and wherein said valve is selected to open at a differential pressure between the inner space of the container and the external ambient of less than 300 mbar during said liquid flow into or from said container.

2. (canceled)

3. A kit according to claim 1 wherein said differential pressure is less than 150 mbar.

4. A kit according to any one of claims 1 or 3 wherein said differential pressure is at least 10 mbar.

5. A kit according to claim 1, wherein less than 25% (v/v) of the total volume of gas contained in said container is exchanged with the external ambient during a period of 6 hours in steady state conditions.

6. A kit according to claim 1, wherein less than 20% (v/v) of the total volume of gas contained in said container is exchanged with the external ambient during a period of 6 hours in steady state conditions.

7. A kit according to claim 1, wherein said second conduit comprises a filtering means for protecting the content of the vial against microbial contamination during liquid withdrawal.

8. A kit according to claim 7, wherein said filtering means comprises a hydrophobic filter.

9. A kit according to claim 1, wherein said first conduit comprises a connecting means for connecting a fluid injector.

10. A kit according to claim 9, wherein said connecting means comprises a luer connector.

11. A kit according to claim 9, wherein said first conduit comprises a shutter means for closing said conduit when said fluid injector is removed.

12. A kit according to claim 1, wherein said valve comprises a bi-directional valve.

13. A kit according to claim 12 wherein said bi-directional valve is a check valve.

14. A kit according to claim 1, wherein said valve comprises at least two mono-directional valves.

15. A kit according to claim 14 wherein said mono-directional valves are spring pressure valves in anti-parallel assembly.

16. A kit according to claim 1, wherein said container comprises a precursor of a suspension of gas-filled microvesicles in the form of a dry lipid deposit in contact with said physiologically acceptable gas.

17. A kit according to claim 16 wherein the suspension of gas-filled microvesicles is obtained by adding a physiological acceptable solution through the first conduit of device.

18. A kit according to any one of claims 1, 5 or 16 wherein said container is a rigid vial comprising a substantially cylindrical body, a flat bottom portion and a top portion defining an open area closed by a stopper hermetically sealing the content of the vial.

19. A kit according to claim 1, further comprising a syringe pre-filled with a physiological acceptable solution.

20. An assembly comprising a combination of the container and the liquid transfer device of claim 1.
